(19) United States
(12) Patent Application Publication
(10) Pub. No.: US 2016/0045357 A1
(43) Pub. Date: Feb. 18, 2016

(54) Shoulder Abduction Support Apparatus

(71) Applicant: SeaWolfe Ventures LLC, Grant, FL (US)

(72) Inventor: George M. Wolfe, Grant, FL (US)

(21) Appl. No.: 14/459,579

(22) Filed: Aug. 14, 2014

(51) Int. Cl. A61F 5/37

(52) U.S. Cl. CPC A61F 5/3753

(57) ABSTRACT

An apparatus for supporting an arm having a support wedge with an essentially triangular prism shape and a shoulder strap attached to the support wedge.

### CROSS REFERENCE TO RELATED APPLICATIONS

This application claims the benefit of provisional patent application Ser. No. 61/866,232, filed with the USPTO on Aug. 15, 2013, which is herein incorporated by reference in its entirety.

### STATEMENT REGARDING FEDERALLY SPONSORED RESEARCH OR DEVELOPMENT

Not applicable.

### INCORPORATION-BY-REFERENCE OF MATERIAL SUBMITTED ON A COMPACT DISK

Not applicable.

### BACKGROUND OF THE INVENTION

1. Field of the Invention

The present invention generally relates to field of post-surgery stabilizer and support devices, more specifically, the present invention relates to a water resistant device used to support and abduct an arm away from the body of a patient while recovering from shoulder surgery.

2. Background Art

Surgery to repair injuries to a patient's shoulder may involve suturing torn tendons and muscles or reattaching tendons to bone. During this surgery, fiber wires may be attached to anchors secured and placed in bone in locations that accommodate and support the binding and suturing together of injured tendons and muscle tissue, so as to allow the tendons and muscle tissue to properly heal. These repairs, and similar repairs, made during surgery may be in a fragile state and are subject to easily being torn loose. To promote a successful surgical recovery, postoperatively the shoulder must remain immobilized in a position as directed by a healthcare provider and for a period of six weeks or longer. This immobilization and positioning enables the muscles and tendons to heal and reattach to bone.

To provide protection and support the arm during the recovery period, healthcare providers may recommend the use of abduction devices. Such devices may support the arm in a particular position so as to allow the muscles to remain in a relaxed state and take tension off of tendons located in the shoulder.

Because the weight of the arm hanging on the shoulder in a non-abducted position, or any motion that could cause shoulder muscles to contract, may cause sutures to tear loose, the healthcare provider may recommend that the patient wear an abduction device at all times during the surgical recovery. Abduction devices which are known in the art utilize pillows to position the arm away from the body in an abducted position. These pillows absorb water and are not suitable for use while the patient showers or bathes. Also, known abduction devices incorporate a sling, in which the forearm is placed, to support the weight of the patient's arm. The sling configuration traps water and is not suitable for use while the patient showers or bathes. Therefore, there is a need in the art for a more versatile abduction device that can be utilized while a patient showers or bathes or at other times when traditional abduction devices are not appropriate.

### BRIEF SUMMARY OF THE INVENTION

The present invention is directed to the idea and methods of and an apparatus for supporting an arm in an abducted position while a patient showers or bathes. One embodiment of the invention may utilize a support wedge positioned under a patent's upper arm. The support wedge may be essentially a triangular prism and comprised of three cylinders made from closed cell polyethylene. The patient's forearm may rest on a face of the support wedge. The support wedge may be connected to a waterproof shoulder strap, which crosses the patient's body and extends over the opposite shoulder. A wrist loop may be connected to the shoulder strap, through which the patient inserts his wrist to allow the wrist loop, in combination with the support wedge, to support the weight of the arm.

### BRIEF DESCRIPTION OF THE DRAWINGS

A better understanding of the present invention will be realized from the detailed description that follows, taken in conjunction with the accompanying drawings, in which:

FIG. 1 depicts an embodiment of the inventive device

FIG. 2 depicts a front view of a patient utilizing the inventive device.

FIG. 3 depicts a rear view of a patient utilizing the inventive device.

### DETAILED DESCRIPTION OF THE INVENTION

Although the following detailed description contains many specifics for the purposes of illustration, anyone of ordinary skill in the art will appreciate that many variations and alterations to the following details are within the scope of the invention. Accordingly, the following preferred embodiments of the invention are set forth without any loss of generality to, and without imposing limitations upon, the claimed invention.

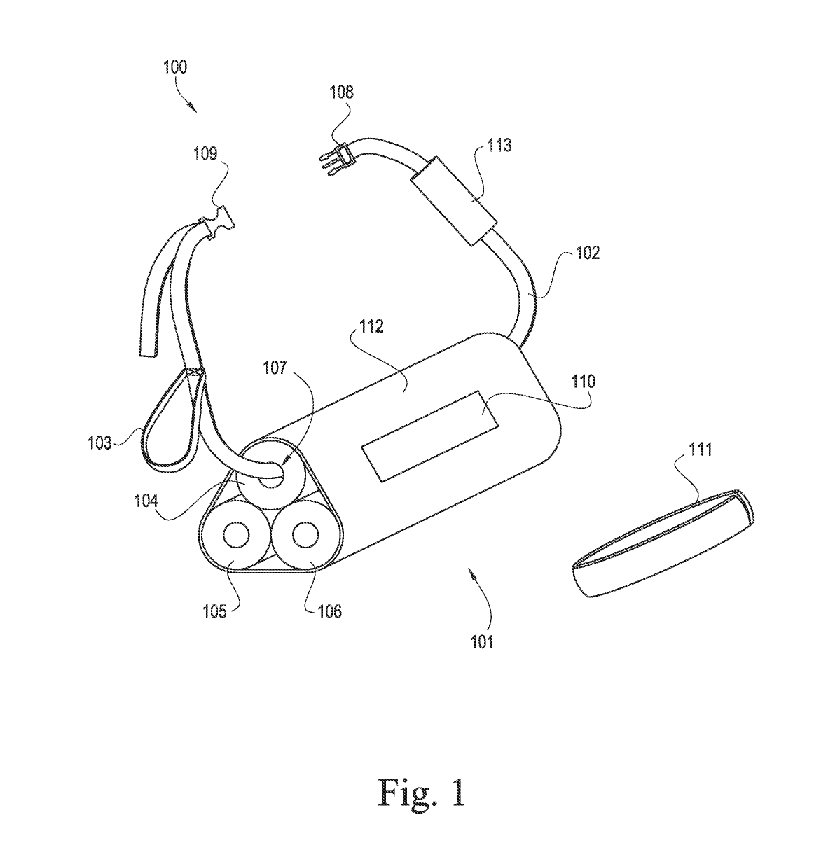

FIG. 1 depicts one embodiment of the apparatus for supporting an arm 100. The apparatus 100 may comprise a support wedge 101. The support wedge 101 may have an essentially triangular prism shape. A shoulder strap 102 may be connected to each of the triangular bases. Alternatively, a shoulder strap 102 may be connected at or near the vertices of the sides of the triangular prism.

In one embodiment, a contact connector 110, such as, by way of example, and not a limitation, one half of a hooks and loops fastener, may be affixed to a face of the support wedge. When worn, the support wedge 101 face with the contact connector 110 may be the face upon which the patient rests his or her forearm for support. In embodiments with the contact connector 110, a sling 111 may have a mating contact connector and may be removably affixed to the contact connector 110 affixed to the support wedge.

The support wedge 101 may be constructed from three cylinders 104, 105, and 106. The three cylinders 104, 105, and 106 may have front faces, which are located toward the front of the patient's body, and rear faces, which are located near the rear of the patient's body, when the apparatus for supporting an arm 100 is donned by the patient. The three cylinders 104, 105, and 106 may form an essentially triangular prism with the left cylinder 105 forming one half of the base of the triangular face and the right cylinder 106 forming the other half of the triangular face. The top cylinder 104 may form the top portion of the triangular face.

These three cylinders 104, 105, and 106 may be covered by a water resistant fabric 112. The three cylinders 104, 105, and 106 wrapped in water resistant fabric 112 may form an essentially triangular prism shape. The three cylinders 104, 105, and 106 may be constructed from a water resistant material. By way of example, and not as a limitation, this water resistant material may be closed cell polyethylene foam.

The top cylinder 104 may have a cavity 107 extending longitudinally through it from the front face to the rear face. Likewise, the left cylinder 105 and right cylinder 106 may have cavities extending longitudinally from their front faces to their rear faces. In one embodiment, the shoulder strap 102 may extend through the cavity 107 of the top cylinder 104.

Each cylinder 104, 105, 106 may be dimensioned such that the cylinder at least twice as long as its diameter. In one embodiment, each cylinder 104, 105, 106 may be at least four times as long as its diameter.

Each cylinder 104, 105, and 106 may have essentially identical dimensions. In such an embodiment, the essentially triangular prism shape may be essentially an equilateral triangular prism shape.

In another embodiment, the support wedge 101 may be constructed from polyethylene foam components, which are not cylinders. By way of example, and not as a limitation, the support wedge 101 may be a single polyethylene component having an essentially triangular prism shape.

In one embodiment, a wrist loop 103 may be attached to the shoulder strap 102. The wrist loop 103 may be configured to support the weight of the patient's arm when the patient's wrist is positioned into the wrist loop 103. The wrist loop 103 may be a continuous strip of material that is permanently affixed to the shoulder strap 102 at both ends of the wrist loop 103. The wrist loop 103 may be affixed to the shoulder strap 102 at a location in front of the support wedge 101. The placement of the wrist loop 103 may be adjustable to ensure proper alignment and positioning of the patient's shoulder and arm.

The shoulder strap 102 may be extend through the support wedge 101, with each end of the shoulder strap 102 joinable to each other by using a rear connector 108 connected to the portion of the shoulder strap extending across the patient's back and a front connector 109 connected to the portion of the shoulder strap extending across the patient's chest. In one embodiment, the shoulder strap may be connected to the rear portion of the support wedge 101 and the front portion of the support wedge 101 and joinable with the use of a front connector 109 and rear connector 108.

A comfort pad 113 may be disposed on the shoulder strap 102. The comfort pad 113 may be configured to move along the length of the shoulder strap 102 and positioned to rest on the patient's shoulder.

The length of the shoulder strap 102 may be adjustable to ensure proper positioning of the support wedge 101 on patients of varying sizes. In some embodiments, the length of the wrist loop 103 may be adjustable.

The wrist loop 103 and the shoulder strap 102 may be made from a water resistant material. By way of example, and not as a limitation, the wrist loop 103 and shoulder strap 102 may be made from polypropylene.

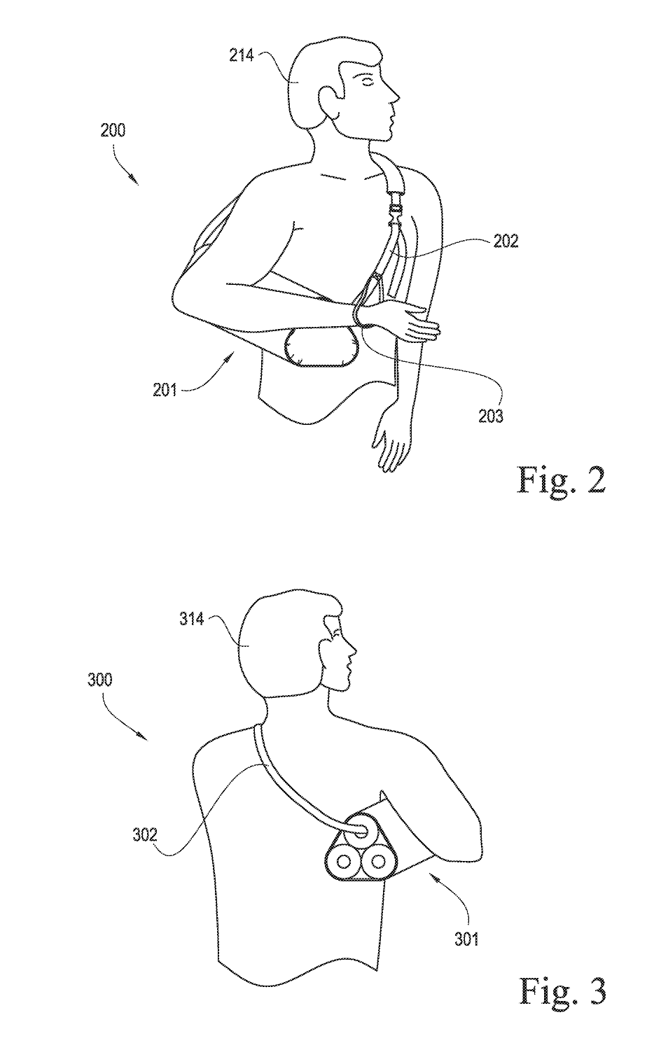

FIG. 2 depicts a front view of the apparatus 200 worn by a patient 214. The shoulder strap 202 extends over the patient's opposite shoulder. The wrist loop 203 and the support wedge 201 support the weight of the patient's arm and maintain it in an abducted position.

FIG. 3 depicts a rear view of the apparatus 300 worn by a patient 314. The shoulder strap 302 extends across the patient's back. The support wedge 301 at least partially supports the weight of the patient's arm and maintains it in an abducted position.

While the above description contains much specificity, these should not be construed as limitations on the scope of any embodiment, but as exemplifications of the presently preferred embodiments thereof. Many other ramifications and variations are possible within the teachings of the various embodiments.

Thus the scope of the invention should be determined by the appended claims and their legal equivalents, and not by the examples given.

What is claimed is:

1. An apparatus for supporting an arm comprising:
a support wedge having an essentially triangular prism shape; and
a shoulder strap attached to the support wedge.

2. The apparatus according to claim 1 further comprising:
a contact connector affixed to the support wedge; and
a sling removably affixed to the contact connector.

3. The apparatus according to claim 1 wherein the support wedge comprises
a top cylinder, having a front face and a rear face;
a left cylinder; and
a right cylinder.

4. The apparatus according to claim 3 further comprising a water resistant fabric covering at least a portion of the top cylinder, the left cylinder, and the right cylinder.

5. The apparatus according to claim 3 wherein the top cylinder has a cavity extending from the front face to the rear face.

6. The apparatus according to claim 5 wherein the shoulder strap extends through the cavity.

7. The apparatus according to claim 3 wherein the top cylinder, the left cylinder, and the right cylinder are constructed from closed cell polyethylene foam.

8. The apparatus according to claim 3 wherein the length of the top cylinder is greater than twice the diameter of the top cylinder.

9. The apparatus according to claim 3 wherein the length of the top cylinder is greater than four times the diameter of the top cylinder.

10. The apparatus according to claim 8 wherein the top cylinder, the left cylinder, and the right cylinder have essentially the same dimensions.

11. The apparatus according to claim 9 wherein the top cylinder, the left cylinder, and the right cylinder have essentially the same dimensions.

12. The apparatus according to claim 1 further comprising a wrist loop attached to the shoulder strap.

13. An apparatus for supporting an arm comprising:
a support wedge;
a shoulder strap attached to the support wedge;
and a wrist loop attached to the shoulder strap.

14. The apparatus according to claim 13 wherein the support wedge has an essentially triangular prism shape.

15. The apparatus according to claim 13 wherein the support wedge comprises a top cylinder, having a front face and a rear face;
a left cylinder; and
a right cylinder.

16. The apparatus according to claim 15 further comprising a water resistant fabric at least partially covering the top cylinder, the left cylinder, and the right cylinder.

17. The apparatus according to claim 15 wherein the top cylinder has a cavity extending from the front face to the rear face.

18. The apparatus according to claim 17 wherein the shoulder strap extends through the cavity.

19. The apparatus according to claim 15 wherein the top cylinder, the left cylinder, and the right cylinder are constructed from closed cell polyethylene foam.

20. The apparatus according to claim 19 wherein the top cylinder, the left cylinder, and the right cylinder have essentially the same dimensions.
